(19) United States
(12) Patent Application Publication
(10) Pub. No.: US 2023/0252775 A1
(43) Pub. Date: Aug. 10, 2023

(54) Sexual Need Interactive Platform System

(71) Applicant: CHIH-FENG LEE, Tainan City (TW)

(72) Inventor: CHIH-FENG LEE, Tainan City (TW)

(21) Appl. No.: 18/004,667

(22) PCT Filed: Jul. 9, 2021
(86) PCT No.: PCT/CN2021/105450

§ 371 Date: Jan. 6, 2023

Related U.S. Application Data
(60) Provisional application No. 63/050,115, filed on Jul. 10, 2020.

(51) Int. Cl. G06V 10/82; G06V 40/20; A61H 19/00

(52) U.S. Cl. CPC G06V 10/82; G06V 40/23; A61H 19/00

(57) ABSTRACT

The present invention is a sexual need interactive platform system, which comprises a content server equipped with a content central processor and a content internal memory for providing a human-machine interaction software; a communication device having a central processor and an internal memory; and a sensor assembly comprising a sensor having 3 or more axes and at least one feedback device, which is electrically connected to the communication device, the sensor having 3 or more axes is worn on an active part of a user for the user to generate a sensor signal, after a user's activity is identified and a classification signal is generated through executing the human-machine interaction software, the classification signal is then converted into a control signal for controlling a content of the human-machine interaction software, and a response generated by the content based on the control signal is transmitted to the feedback device for feed-backing a result of the user's activity, wherein the content server and the communication device are interconnected through an internet network.

### TECHNICAL FIELD

The present invention relates to a sexual need interactive platform system, particularly one that can use artificial intelligence technology to analyze sensor data, precisely responds to an interactive effect of user behavior and content, enjoys high-quality virtual love; and produces near-infinite, high-quality video and audio as the material of system content.

### BACKGROUND OF THE INVENTION

Traditionally, adult content such as pictures, films are usually presented as one side of view. A few contents such as games can allow users to control in a limited way through a standard human-machine interface (such as a keyboard or a mouse). Since the use process cannot interact more intuitively with content roles, it is uninteresting, boring and not attractive.

### DETAILED DESCRIPTION OF THE INVENTION

The main purpose of the present invention is to provide a sexual need interactive platform system to overcome the above shortcomings of conventional technology, comprising a content server equipped with a content central processor and a content internal memory for providing a human-machine interaction software, which is selectable random contents; a communication device having a central processor and an internal memory, wherein the central processor is used for executing the human-machine interaction software to allow a user to perform sexual need exercises with executed the selectable random contents so as to control the sexual need interactive platform system; and a sensor assembly comprising a sensor having 3 or more axes and at least one feedback device, which is electrically connected to the communication device, wherein the sensor having 3 or more axes is worn on an active part of the user for the user to generate a sensor signal, the sensor signal is transmitted to the communication device, and a user's activity is identified and a classification signal is generated through executing the human-machine interaction software, and then the classification signal is converted into a control signal for controlling a content (including roles, scenes and partners) of the human-machine interaction software, a response generated by the content based on the control signal is transmitted to the feedback device for feed-backing a result of the user's activity, wherein the content server and the communication are interconnected through an internet network.

A sexual need interactive platform system, wherein the feedback device is a vibration motor or an activity structure.

In one embodiment, a sexual need interactive platform system of the present invention further comprises another software program executed in the central processor, connecting the sensor assembly and the human-machine interaction software for transmitting the control signal corresponding to a sensor classification result to the human-machine interaction software, and for transmitting one feedback signal of the content of the human-machine interaction software to the sensor assembly, so as to bidirectionally transmit the control signal between the sensor assembly and the human-machine interaction software, thereby assisting the user in achieving a bilateral control effect.

In one embodiment, a sexual need interactive platform system of the present invention, wherein the human-machine interaction software comes from a network, a compact disk or a flash drive or other storage devices.

In one embodiment, a sexual need interactive platform system of the present invention, wherein the human-machine interaction software is a software that performs exercises simulating real sex.

In one embodiment, a sexual need interactive platform system of the present invention further comprises a video activity analysis software, wherein after any sexual activity video such as a film, live streaming is analyzed by the video activity analysis software, all sexual behaviors within the analysis range are recovered in real time to sexual activities through controlling the appropriate sensor assembly by the video activity analysis software, allowing sexual organs of the user to experience real feeling like a main role in the film.

In one embodiment, a sexual need interactive platform system of the present invention further comprises an audio-visual film playback software for storing audio-visual data and classification parameters on the communication device, wherein when wearing the sensor assembly, hand gesture/control identification is performed by transmitting the sensor signal to the audio-visual playback software to achieve the effect of enhancing interaction with the film.

In one embodiment, a sexual need interactive platform system of the present invention, wherein the assembly having a sensor having 3 or more axes and a feedback device is a first sensor assembly, accelerations, movement trajectories and positions are detected through installation of a nine-axis sensor (a triaxial accelerometer, a triaxial gyroscope, a triaxial magnetometer) wristband, which are used to determine directions, angles, speeds and postures, in addition, it has control detection and gesture identification functions, and the feedback device is used for feed-backing a user's activity result.

In one embodiment, a sexual need interactive platform system of the present invention, wherein the assembly having a sensor having 3 or more axes and a feedback device is a second sensor assembly, it is used as a sensor for AR augmented reality through a position marker.

In one embodiment, a sexual need interactive platform system of the present invention, wherein the assembly having a sensor having 3 or more axes and a feedback device is a third sensor assembly, which was used as a VR controlling device, an electromyography sensor (EMG) is additionally added, electromyographic signals are analyzed through an artificial intelligence algorithm to realize various gestures and operations as fine as movements of ten fingers, which was used in the VR version of the sexual need interactive platform system of the present invention.

The sexual need interactive platform system according to the first embodiment of the present invention further comprises an artificial intelligence server electrically connected to the Internet network, the artificial intelligence server has an artificial intelligence central processor and an artificial intelligence internal memory, wherein an artificial intelligence training software is executed on the artificial intelligence central processor, and the artificial intelligence training software has a machine learning, a deep learning, a natural language and an audio conversation training functions, and trained parameters are used for various content demands of the platform; wherein the artificial intelligence training software adopts the generative adversarial network (GAN) technology, which is a role generation adversarial network module for being used in a platform role generation module, and generates virtual roles through mass audio-visual data trainings; and an artificial intelligence classification technology, which comprises a sensor signal classification module, wherein after a sensor signal is collected in a training stage, an artificial intelligence deep learning result is used for classifying the sensor signal to generate a classification parameter, used for receiving the sensor signal in an inference stage so as to obtain a behavior classification of the user, and then the behavior classification and the content of the human-machine interaction software are combined to achieve an interactive and intuitive control manner; and also comprises a video activity analysis module, which analyzes sexual activities of the roles in a video (for example, handjob, oral sex and sexual intercourse), can convert a sexual activity process into control signals of the vibration motor and the activity structure, so that the sensor assembly generates activities of the handjob, oral sex and sexual intercourse as in the screen to allow the user to experience a sexual feeling like a main role in the screen.

The sexual need interactive platform system according to the first embodiment of the present invention, wherein the role generative adversarial network module further comprises an infinite audio generation sub-module which adopts Cycle GAN audio technology to simulate the voice of a first timbre by using a second timbre; and an infinite video role generation sub-module for generating various roles required by the system.

The sexual need interactive platform system according to the first embodiment of the present invention further comprises an intelligent digital lens electrically connected to the artificial intelligence server for face identification, emotion identification, behavior identification and age identification to generate a multi-modal output value, and a past video data in the artificial intelligence server is used to perform an artificial intelligence calculation to generate an optimal physiological activity condition signal.

The sexual need interactive platform system according to the first embodiment of the present invention further comprises an intelligent speaker device electrically connected to the artificial intelligence server, wherein the intelligent speaker device converses with the user after receiving a voice-frequency conversation signal of the user from the artificial intelligence server, and then performs an artificial intelligence calculation to generate a first multimodal output value and a first identification voice-frequency conversation signal.

The sexual need interactive platform system according to the first embodiment of the present invention further comprises a user preference and habit identification server, electrically connected to the artificial intelligence server and having a user preference and habit identification central processor and a user preference and habit identification internal memory, wherein a user preference and habit identification software is executed on the user preference and habit identification central processor, for recording the user's behaviors, performing an artificial intelligence calculation and then generating a user sexual preference and habit identification signal, so that the user preference and habit identification server generates a recommendation system based on the user's preferences and habits.

The sexual need interactive platform system according to the first embodiment of the present invention further comprises an audio-video playback software, which allows the users to self-define various sex video clips (for example, insertion, normal position, back position, woman on top position, orgasm, etc.), when wearing the sensor to masturbate, the sensing signal is transmitted to the software through an activity to perform gesture/control identification, so as to quickly switch the sexual video clips according to the user's thinking, or control the video clip speed, or receive a video clip feedback, enhancing the effect of interacting with the roles in the film.

The sexual need interactive platform system according to the first embodiment of the present invention further comprises a physician robot computer device, electrically connected to the user preference and habit identification server and having a physician robot central processor and a physician robot internal memory, which is used for managing and monitoring the sexual need system while monitoring the user's physiological and medical data, so as to generate a physician robot diagnostic signal after performing an artificial intelligence calculation, and the user is informed with a sexual health diagnostic result through the intelligent speaker device.

The sexual need interactive platform system according to the first embodiment of the present invention, wherein the multi-modal output value is output values of a face identification status, an emotion identification status, a behavior identification status and an age identification status.

The sexual need interactive platform system according to the first embodiment of the present invention, wherein when the user wears the sensor assembly, it is electrically connected to a user personal health data server for transmitting the user's physiological data sensed by the sensor assembly to the user personal health data server.

The sexual need interactive platform system of the first embodiment of the present invention, wherein the sexual need interactive platform system can be switched among an artificial intelligence virtual role state, an artificial intelligence optimal sexual activity condition state and an artificial intelligence automatic sexual diagnostic state.

A sexual need interactive platform system according to the second embodiment of the present invention comprises a sexual need interactive platform system, comprising a content server equipped with a content central processor and a content internal memory for providing a human-machine interaction software, which is selectable random contents; a first communication device, having a first communication device central processor and a first communication device internal memory, wherein the first communication device central processor is used for executing the human-machine interaction software to allow a first user to perform sexual need exercises according to selectable random contents so as to control the sexual need interactive platform system; a first sensor assembly which comprises a first sensor having 3 or more axes, a first vibration motor and a first activity structure, electrically connected to the first communication device, wherein the sensor having 3 or more axes is worn on an active part of a first user for the first user to generate a first user activity signal, the first user activity signal is converted into a first user activity classification signal on the central processor of the first communication device, and then converted into a first activity control signal so as to be transmitted to an internet network by using the first communication device; a second communication device electrically connected to the first communication device, and having a second communication device central processor and a second communication device internal memory, wherein the second communication device central processor is used for executing the human-machine interaction software to allow a second user to perform sexual demand exercises according to the selectable random contents so as to control the sexual need interactive platform system; and a second sensor assembly which comprises a second sensor having 3 or more axes, a second vibration motor and a second activity structure, which is electrically connected to the second communication device, wherein the sensor having 3 or more axes is worn on an active part of the second user for the second user to generate a second user activity signal, the second user activity signal is converted into a second user activity classification signal on the central processor of the second communication device, and then converted into a second activity control signal so as to be transmitted to the internet network by using the second communication device, wherein the first communication device receives the second activity control signal from the internet network and then recovers the original activity of the second user through the first activity structure; or the second communication device receives the first activity control signal from the internet network and then recovers the original activity of the first user through the second activity structure, wherein the content server, the first communication device and the second communication device are connected via the internet network

A sexual need interactive platform system according to the third embodiment of the present invention, which comprises: a communication device having a central processor and an internal memory for controlling the sexual need interactive platform system; a human-machine interaction software for providing human-machine interface interaction, when being loaded on the communication device, the central processor is used for executing the human-machine interaction software to allow a user to interact with the human-machine interaction software; and a sensor assembly comprising a sensor having 3 or more axes and at least one feedback device, which is electrically connected to the communication device, wherein the sensor having 3 or more axes is worn on an active part of the user for the user to generate a sensor signal, the sensor signal is transmitted to the communication device, a user's activity is identified and a classification signal is generated through executing the human-machine interaction software, and then the classification signal is converted into a control signal for controlling a content of the human-machine interaction software (including roles, scenes and partners, etc.), a response generated by the content based on the control signal is transmitted to the feedback device for feed-backing a result of the user's activity.

A sexual need interactive platform system of the present invention, wherein the communication device and the sensor assembly are interconnected in a wired or wireless (for example, Bluetooth, WIFI) manner.

The “communication device” of the present invention generally refers to a computer device, a mobile phone, a PDA or other devices having processing, storage and display.

### BRIEF DESCRIPTION OF THE DRAWINGS

FIG. 1 is a schematic diagram of the structure of the sexual need interactive platform system according to the first embodiment of the present invention.

FIG. 2A is a schematic diagram of the structure of the sexual need interactive platform system according to the second embodiment of the present invention.

FIG. 2B is a schematic diagram of the structure of the sexual need interactive platform system according to the third embodiment of the present invention.

FIGS. 3A and 3B are schematic diagrams of the first wearing state of the first sensor assembly of the present invention.

FIGS. 4A and 4B are schematic diagrams of the second wearing state of the first sensor assembly of the present invention.

FIGS. 5A and 5B are schematic diagrams of the states of use of the first sensor assembly of the present invention.

FIGS. 6A to 6C are schematic diagrams of the application methods of the present invention.

FIGS. 7A and 7B are schematic diagrams of the states of use of the second sensor assembly of the present invention.

FIG. 8A is a schematic diagram of the wearing state of the third sensor assembly of the present invention.

FIG. 8B is a schematic diagram of the state of use of the third sensor assembly of the present invention.

### EXAMPLES

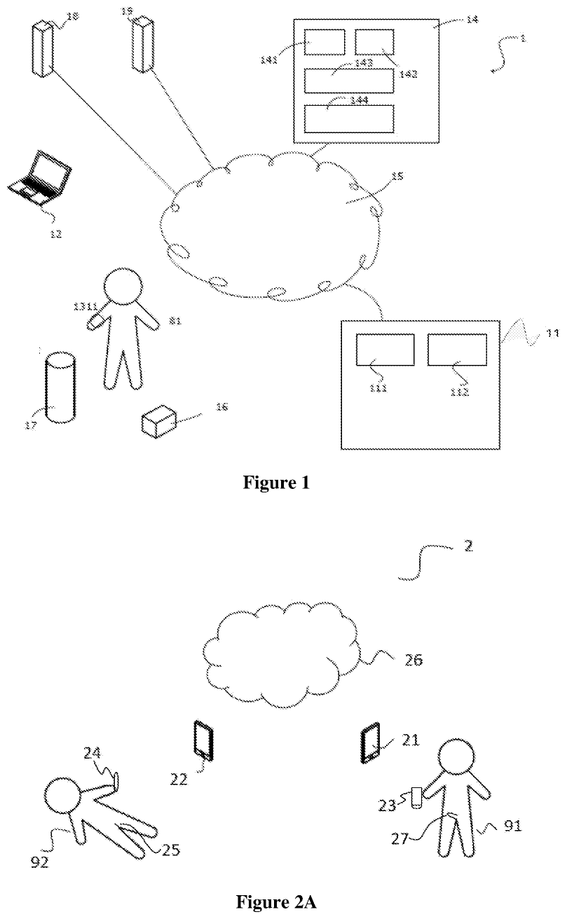

As shown in FIG. 1, the sexual need interactive platform system 1 according to the first embodiment of the present invention comprised: a content server 11 having a content central processor 111 and a content internal memory 112 for providing a first user 81 a human-machine interactive software, which comprised selectable contents on demand, which comprised videos, audios or operation scenes, to provide the first user 81 with related contents for operating the system; a communication device 12 having a central processor and an internal memory, wherein the central processor was used for executing the human-machine interaction software to allow the first user 81 to perform sexual need exercises according to selected contents, thereby managing and controlling the sexual need interactive platform system 1; and a sensor assembly 1311, comprising an assembly having a sensor having 3 or more axes and a vibration motor for the first user 81, electrically connected to the user communication device 12, the sensor having 3 or more axes was worn on an active part of the first user 81 (for example, the user's limb or a movable joint) to allow the first user 81 to generate a sensor signal, to interact with the first user 81 through executing and operating the human-machine interaction software, and the vibration motor fed back an activity result of the first user 81.

The sexual need interactive platform system 1 according to the first embodiment of the present invention further comprised an artificial intelligence server 14, electrically connected to an internet network 15, wherein the artificial intelligence serve 14 was provided with an artificial intelligence central processor 141 and an artificial intelligence internal memory 142, an artificial intelligence training software 143 was executed on the artificial intelligence central processor 141, and the artificial intelligence training software 143 had machine learning and deep learning and natural language and audio conversation training functions, and trained parameters were used for various content demands of the platform; the artificial intelligence training software 143 adopted generative adversarial network (GAN) technology, which was a role generative adversarial network module for being used in a platform role generation module, and generated virtual roles through mass audio-visual data trainings; and an artificial intelligence classification technology for being used in a sensing signal classification module, after the sensor signal was collected in a training stage, an artificial intelligence deep learning result was used for classifying the sensor signal, a classification parameter was generated for receiving the sensor signal in an inference stage so as to obtain a user behavior classification, and then the behavior classification and the content of the human-machine interaction software were combined to achieve an interactive and intuitive control manner; and also comprised a video activity analysis module, which analyzed sexual activities of the roles in a video (for example handjob, oral sex and sexual intercourse), and could convert a sexual activity process into control signals of the vibration motor and the activity structure.

The sexual need interactive platform system 1 according to the first embodiment of the present invention, wherein the role generative adversarial network module comprised an infinite audio generation sub-module which adopted Cycle GAN audio technology to simulate the voice of a first timbre by using a second timbre (could be real or synthetic timbre); and an infinite video role generation sub-module for generating various roles required by the system by using the GAN.

The sexual need interactive platform system 1 according to the first embodiment of the present invention, further comprised an intelligent digital lens 16, electrically connected to the artificial intelligence server 14 for a video identification software 144 to perform face identification, emotion identification, behavior identification and age identification, so as to perform an artificial intelligence calculation and generate an optimal sexual condition signal with a past video data in the artificial intelligence server.

The sexual need interactive platform system 1 according to the first embodiment of the present invention further comprised an intelligent speaker device 17, electrically connected to the artificial intelligence server 14, wherein the intelligent speaker device 17 conversed with the user after receiving an audio conversation signal of the user from the artificial intelligence server 14, and then generated a first multimodal output value and a first identification voice-frequency conversation signal after performing an artificial intelligence calculation.

The sexual need interactive platform system 1 according to the first embodiment of the present invention further comprised a user preference and habit identification server 18, electrically connected to the artificial intelligence server 14, and having a user preference and habit identification central processor and a user preference and habit identification internal memory, wherein a user preference and habit identification software was executed on the user preference and habit identification central processor for recording the user's behaviors, performing an artificial intelligence calculation and then generating a user sexual preference and habit identification signal, so that the user preference and habit identification server 18 could generate a recommendation system based on the user's preferences and habits.

The sexual need interactive platform system 1 according to the first embodiment of the present invention further comprised an audio-visual playback software for storing audio-visual data and classification parameters on an optical disc drive, a flash drive or any storage device of the communication device, or for downloading sexual audio-visual data and classification parameters in the communication device 12 by the content server, or for self-defining viewing video clips by the user, wherein the source of the film could be existing films (from a network, a compact disk or any storage device), or random films of the content server, wherein when the user wore the sensor assembly 1311, the sensor signal was transmitted to the audio-video playback software through the user's activities, and hand gesture/control identification was performed to achieve the effect of fast switching sexual clips, controlling the film speed, or receiving a film feedback and enhancing interaction with the roles and contents of the film.

The sexual need interactive platform system 1 according to the first embodiment of the present invention further comprised a physician robot computer device 19, electrically connected to the user preference and habit identification server 18, and having a physician robot central processor and a physician robot internal memory, which was used for managing and monitoring the sexual need system while monitoring the user's physiological and medical data, so as to generate a physician robot diagnostic signal after performing an artificial intelligence calculation, and the user was informed with a sexual health diagnostic result through the intelligent speaker device 17.

In one embodiment, a sexual need interactive platform system 1 of the present invention, wherein the assembly having a sensor having 3 or more axes and at least one feedback device was a first sensor assembly, accelerations, movement trajectories and positions were detected through installation of a nine-axis sensor (a triaxial accelerometer, a triaxial gyroscope, a triaxial magnetometer) wristband, which were used to determine directions, angles, speeds and postures, in addition, it had control detection and gesture identification functions, and the feedback device was used for feed-backing a user's activity result.

The sexual need interactive platform system 1 according to the first embodiment of the present invention, wherein the assembly having a sensor having 3 more axes and at least one feedback device was a second sensor assembly, which was used as a sensor for AR augmented reality, a positioning mark was installed on the second sensor assembly so as to bring the role video to a three-dimensional space through a mobile device, a tablet computer, an e-sports computer or other display devices (for example AR glasses).

The sexual need interactive platform system 1 according to the first embodiment of the present invention, wherein the assembly having a sensor having 3 or more axes and at least one feedback device was a third sensor assembly, which was used as a VR controlling device, an electromyography sensor (EMG) was additionally added, electromyographic signals were analyzed through an artificial intelligence algorithm to realize various gestures and operations as fine as movements of ten fingers, which was used in the VR version of the sexual need interactive platform system of the present invention.

As shown in FIG. 1, the sexual need interactive platform system 1 according to the first embodiment of the present invention, in an interactive mode, a first user 81 used a sensor assembly 1311 having a sensor having 3 or more axes and at least one feedback device to control the sexual need interactive platform system 1, the selectable random sexual contents were transmitted by the content server 11 to the communication device 12 of the first user, allowing the first user 81 to interact with various displayed roles/videos/software.

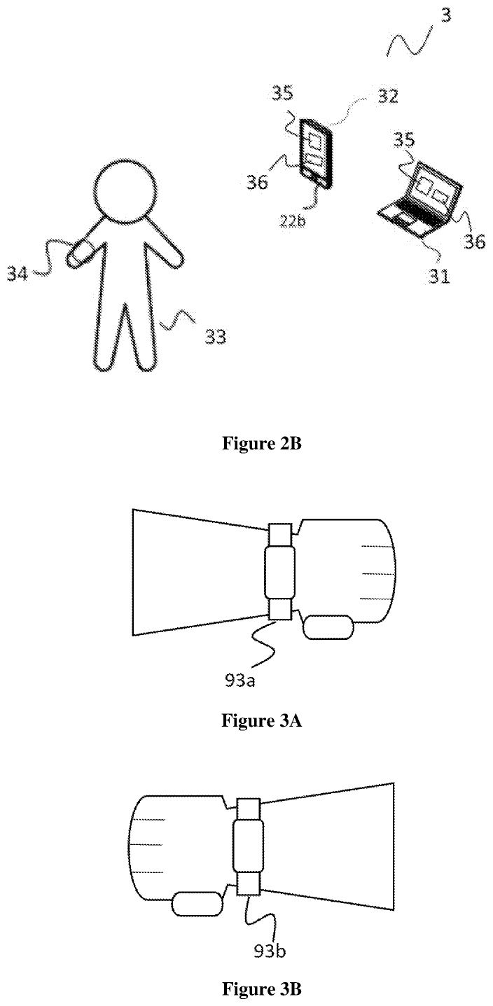

As shown in FIG. 2A, the sexual need interactive platform system 2 according to the second embodiment of the present invention, which comprised: a content server equipped with a content central processor and a content internal memory for providing a human-machine interaction software, which comprised selectable contents on demand; a first communication device 21 having a first communication device central processor and a first communication device internal memory; a first sensor assembly 23 having an artificial vagina function, electrically connected to the first communication device 21 for allowing a male user 91 to generate a male user sensor signal, and the male user sensor signal was converted into a first activity classification signal on the first communication device central processor, then converted into a first activity control signal so as to be transmitted to an internet network 26 by the first communication device 21; a second communication device 22, connected to the first communication device 21 via the internet network 26, and having a second communication device central processor and a second communication device internal memory; and a second sensor assembly 24 having an artificial penis function, electrically connected to the second communication device for allowing a female user 92 to generate a female user sensor signal, the female user sensor signal was converted into a second activity classification signal on the second communication device central processor, and then converted into a second activity control signal, for being transmitted to the first communication device 21 by the second communication device 22 via the internet network 26, so that the first communication device 21 could receive the second activity control signal of the female user 92, and recovered the original activity of the second user 92 through the first sensor assembly 23 having an artificial vagina function of the male user, and vice versa; this system was used to recover activity details between users and enhanced the real feeling of long-distance interaction. For example, the male user 91 put on a first sensor assembly 23 having an artificial vagina (masturbation cup) function on the male sex organ 27 of the male user, the female user 92 wore a second sensor assembly 24 having an artificial penis function, and the two sensor assemblies (23, 24) were interlinked through the communication devices (21, 22) via the internet network 26 to conduct long-distance sexual intercourse, when the second sensor assembly 24 having an artificial penis function was put into the female sexual organ 25 of the female user 92 for operation, the first sensor assembly 23 having the artificial vagina function responded in real time the activity of the female sexual organ 25 of the female user 92 to the second sensor assembly 24 having an artificial penis function, the activity was recovered to the male organ 27 of the male user 91 through the first sensor assemble 23 having an artificial vagina function, and vice versa.

Both parties could feel excitements of the sexual behaviors of each other, and achieved a near-real long-distance sex, the method of which could also be broadcasting, for example, a male user broadcasted by using the first sensor assembly 23 having an artificial vagina function, a group of female users used the second sensor assembly 24 having an artificial penis function to respectively link with the first sensor assembly 23 having an artificial vagina function to synchronously feel the pleasure of having virtual sex with the male; alternatively, a female could be equipped with the second sensor assembly 24 having an artificial penis function, and allowed a group of males to respectively use the first sensor assembly 23 having an artificial vagina function and link to the second sensor assembly 24, the first sensor assembly 23 having an artificial vagina function reflected the activity of the female to the second sensor assembly 24 having an artificial penis function, so that the penises of the group of males could experience nearly real-time sexual feelings.

As shown in FIG. 2B, the sexual need interactive platform system 3 according to the third embodiment of the present invention, which comprised: a first communication device 31 or a portable device 32 (such as a mobile device or a tablet), having a first communication device central processor and a first communication device internal memory; a human-machine interaction software 35 was installed on a first communication device, and another software program 36 was run on the first communication device, a human-machine interaction software 35 could come from any storage device such as a network, a compact disc, a flash drive, wherein a sensor assembly 34 comprised a sensor having 3 or more axes and at least one feedback device, electrically connected to the first communication device for allowing a user 33 to generate a user activity signal, which was transmitted to the communication device 31 or the portable device 32, a classification signal was generated after another software program 36 identified the activity of the user 33, and then converted into a control signal to control the content of the human-machine interaction software 35, when a feedback was generated by the content based on the user's control, the feedback information was transmitted to another software program 36, and converted into a control signal of the feedback device of the sensor assembly by another software program 36, so as to bi-directionally transmit the control signal to the sensor assembly 34 and the human-machine interaction software 35, thereby achieving the effect of feed-backing the user 33 by the human-machine interaction software 35 according to the result by using the feedback device of the sensor assembly 34.

FIG. 3A and FIG. 3B are schematic diagrams of the first wearing state of the first sensor assembly of the present invention, wherein the first sensor assemblies (93a, 93b) were worn by one hand or both hands in pairs, the first wearing state was equipped with an accelerator having 3 or more axes and a vibration motor, the function of the vibration motor was to transmit system software feedback responses, such as event notification. The first sensor assembly 93a was worn on the left hand, and the other first sensor assembly 93b was worn on the right hand.

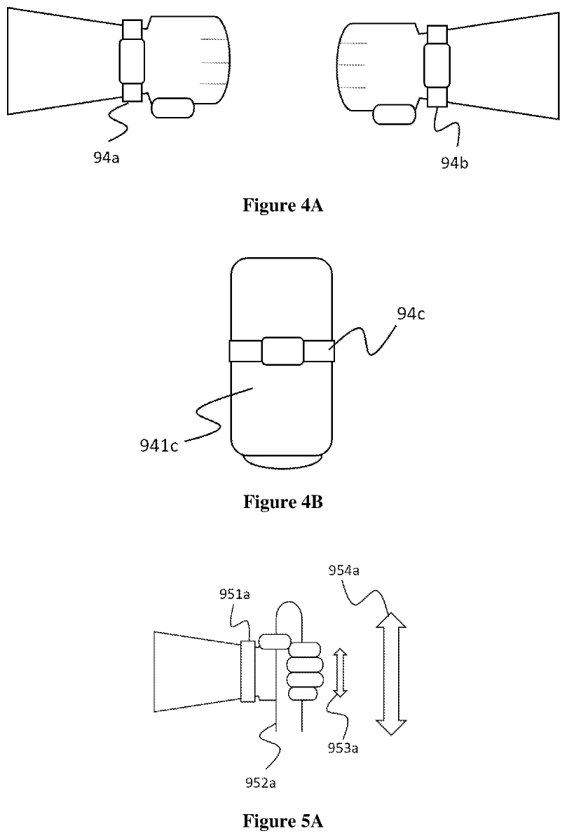

The sexual need interactive platform system of the present invention, wherein the first sensor assembly was provided with a nine-axis sensor (a triaxial accelerometer, a triaxial gyroscope, a triaxial magnetometer) wristband capable of detecting accelerations, movement trajectories and positions, which were used to determine directions, angles, speeds and postures, in addition, it had an activity detection function. When the sensor assembly was worn on the wrist, activities of the palm or wrist could be calculated (gesture determination), which became an input signal for interacting with the contents on demand of the system. The sensor could be worn on the wrist, as shown in FIG. 4A, or the sensor could also be fixed onto any devices by using a fastener, as shown in FIG. 4B.

FIG. 4A and FIG. 4B are schematic diagrams of the second wearing state of the first sensor assembly of the present invention, wherein there were a total of three first sensor assemblies (94a, 94b, 94c), the second wearing state was equipped with an accelerator having 3 or more axes and a vibration motor, the function of the vibration motor was to transmit a feedback of the human-machine interaction software, for example, the roles' responses, the first sensor assembly (94a, 94b, 94c) were respectively assembled on the left hand, the right hand and fixed onto a device for use, wherein the first sensor assembly 94a was worn on the left hand, and the second sensor assembly 94b was worn on the right hand, the left and right hands were used for controlling foreplay and role interactions; the third first sensor assembly 94c was fixed onto a device such as a masturbation cup 941c, and used for sex and body positions related to interactions with the roles after insertion.

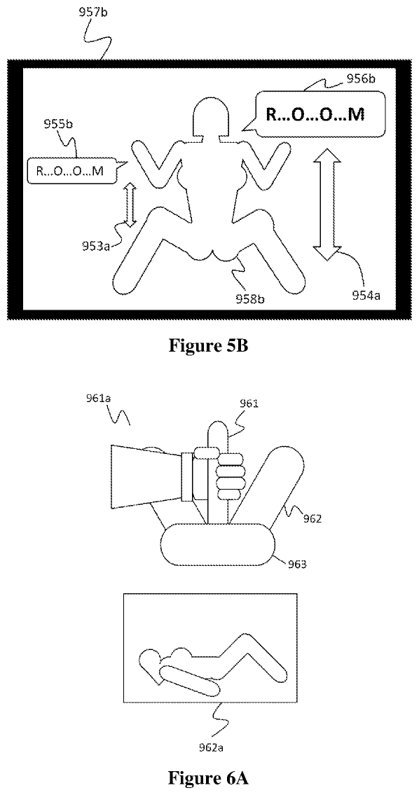

FIG. 5A and FIG. 5B are schematic diagrams of the state of use of the first sensor assembly of the present invention, when the user wore a wristband sensor 951a on his hand and held the penis 952a with his hand, he performed a slow masturbation activity 953a or a fast masturbation activity 954a, the role 958b on the computer monitor 957b had a relatively small shaking response under the slow masturbation activity 953a, and the sound of the female copulation call was relatively low, as shown in the platform female role sound indication block 955b. Under the condition of fast masturbation activity 954a, the platform female 958b had a relatively big shaking response, and the sound of the female copulation call was louder, as shown in the sound indication block 956b of the female role on the platform.

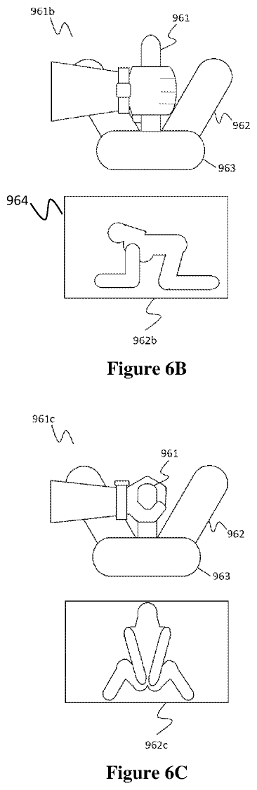

FIG. 6A to FIG. 6C are the embodiments of the application method of the present invention, which are shown in Table 1

TABLE 1
Example of the application method of
the sensor assembly on a wristband

| Scene | | | |
|---|---|---|---|
| Function | Role | Role | Playback |
| Gesture | interaction 1 | interaction 2 | procedure |
| Palm facing up, | Controlling | 962a Role on | Skipping |
| forward and | the role to | missionary | to clip A |
| backward 961a | lie on the | body position | |
| | back | (FIG. 6A) | |
| Palm facing down, | Controlling | 962b Role on | Skipping |
| forward and | the role to | back body | to clip B |
| backward 961b | lie down | position | |
| | | (FIG. 6B) | |
| Purlicue facing | Controlling | 962c Role on | Skipping |
| down, upward and | the role to | riding body | to clip C |
| downward 961c | sit facing | position | |
| | forward | (FIG. 6C) | |
| Purlicue facing | Controlling | Role on back | Skipping |
| down, upward and | the role to | riding body | to clip D |
| downward | sit with | position | |
| | back facing | | |
| | forward | | |
| Big activity | Big activity | Running forward | Skipping |
| turning to | right | | to next clip |
| the right | operation | | |
| | from cursor | | |
| Small activity | Small | Walking forward | Fast forward |
| turning to | activity | | |
| the right | right | | |
| | operation | | |
| | from cursor | | |
| Big activity | Big activity | Running backward | Skipping to |
| turning to | left operation | | previous clip |
| the left | from cursor | | |
| Small activity | Small | Walking backward | Fast backward |
| turning to | activity left | | |
| the left | operation | | |
| | from cursor | | |
| Moving forward | Caress | Piston movement | Normal |
| and backward | operation | | speed/fast |
| | from cursor | | speed/slow |
| | | | speed playback |
| Palm facing | Confirming | Insertion | Start playback |
| down, rotating | activity | | |
| 180 degree | | | |
| Palm facing | Cancel | Pulling out | Stop playback |
| up, rotating | activity | | |
| 180 degree | | | |

*: This table describe examples, and actual applications are not limited to what are described in this table.
**: 964 is user display device

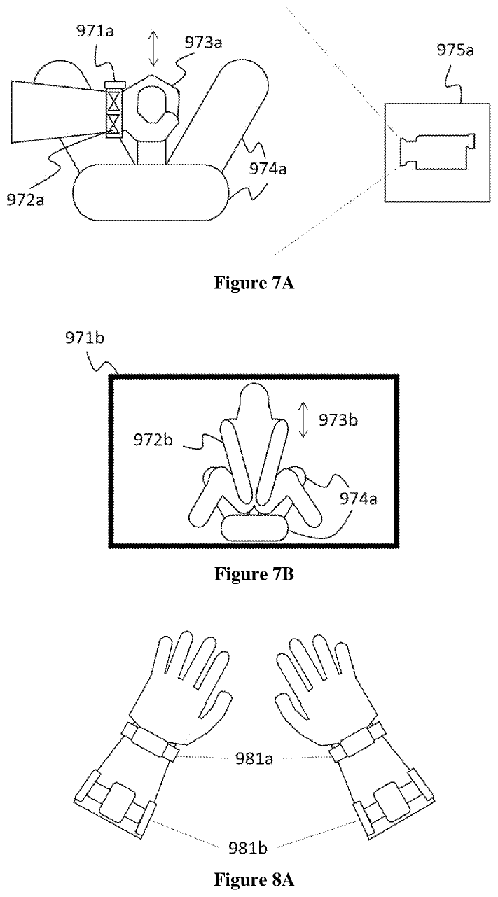

As shown in FIG. 7A and FIG. 7B, the sexual need interactive platform system of the present invention, wherein the second sensor assembly 971a was used as a sensor for AR augmented reality, and a positioning mark 972a was arranged on the second sensor assembly 971a to execute the AR version of the sexual need interactive platform system of the present invention. As an example shown in the drawing, when the user held the penis with his hand for a up and down movement 973a, the AR augmented reality sensor 975a (for example, a camera) took pictures, and the AR display 971b (for example, display devices such as a mobile device or a tablet computer, an e-sports computer or AR glasses, etc.) displayed the AR partner-AR female role 972b who appeared on the user's body in a riding position on the screen through the positioning mark 972a. When the user held the penis for a movement, the AR partner-AR female role 972b could move up and down 973b following the movement of the positioning mark 972a, as if the user was really having sex with a woman, thereby bringing the video to a three-dimensional space and enhancing the user's experience.

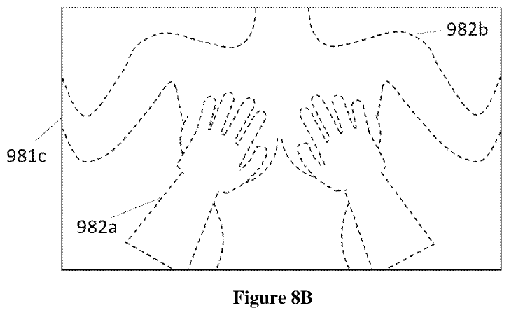

As shown in FIG. 8A and FIG. 8B, the sexual need interactive platform system of the present invention, wherein the assembly having a sensor having 3 or axes and a vibration motors was a third sensor assembly 981a, the third sensor assembly 981a was a VR game controller, and an electromyography sensor 981b was further added, an artificial intelligence algorithm was used to analyze the electromyographic signal to realize the operation of fine movements. A conventional VR controller was of a hand-held type, and the user must reserve several fingers to hold the controller so as to prevent it from falling off. The present invention allowed ten fingers of both hands to be completely free so as to perform an all-finger operation. As shown in FIG. 8A, the user wore the sensor assembly 981a and the electromyography sensor 981b, for being used in the VR version of the sexual need interactive platform system of the present invention, the user's VR arm 982a was displayed on the VR display 981c, as a result, ten fingers could be used to touch and stroke the VR female role 982b and perform fine finger operations.

The first sensor assembly used by the sexual need interactive platform system of the present invention adopted AIoT technology, an artificial intelligent machine learning technology was used to perform interactive gesture process classification, a training software was further included, the training software adopted the generative adversarial network (GAN) technology, a content generative adversarial network module generated various contents and roles. The content generative adversarial network module was used to generate content and role trainings through a large number of videos. The content generative adversarial network module comprised an infinite audio generation sub-module, which was used to use Cycle GAN audio technology to simulate the voice of A timbre by using a B timbre; and an infinite video content generation sub-module, which was used to generate various contents by using GAN; and an artificial intelligence classification technology, which comprised a sensor signal classification module used for receiving a sensor signal and inputting into the sensor signal classification module, and an artificial intelligence deep learning was used to classify the sensor signal to generate a classification signal. It also comprised a video activity analysis module for analyzing sexual activities of the roles in the video (such as handjob, oral sex, and sexual intercourse), and a sexual activity process could be converted into control signals of the vibration motor and the activity structure, so that the assembly generated the activities such as handjob, oral sex, and sexual intercourse like those in the screen, allowing the user to experience the feeling of sex like the main role in the screen.

The extended application of the infinite audio generation sub-module was a role-dubbing sub-module in which one person played multiple roles, it could be performed by one person and dubbed by multiple artists. The application scenarios include dubbing artists, storytellers, etc. The content generative adversarial network module was a content material generation device that could synthesize a variety of contents.

### SYMBOL DESCRIPTION

1—sexual need interactive platform system

11—content server

111—content central processor

112—content internal memory

12—communication device

1311—sensor assembly

14—artificial intelligence server

141—artificial intelligence central processor

142—artificial intelligence internal memory

143—training software

144—video identification software

15—internet network

16—intelligent digital lens

17—intelligent speaker device

18—user preference and habit identification server

19—physician robot computer device

2—sexual need interactive platform system of the second embodiment

21—first communication device

22—second communication device

23—first sensor assembly having an artificial vagina function

24—second sensor assembly having an artificial penis function

25—female sex organ

26—internet network

27—male sex organ

3—sexual need interactive platform system of the third embodiment

31—first communication device

32—portable device

33—user

34—sensor assembly

35—human-machine interaction software

36—another software program

81—first user

91—male user

92—female user

93a—first sensor assembly

93b—another first sensor assembly

94a—first first sensor assembly

94b—second first sensor assembly

94c—third first sensor assembly

941c—masturbation cup

951a—wristband sensor

952a—penis

953a-954a—masturbation activity

955b-956b—platform female role sound indication block

957b—computer monitor

958b—platform female role

961—user's penis

961a—wearing sensor assembly, palm facing up and moving forward and backward

961b—wearing sensor assembly, palm facing down and moving forward and backward

961c—wearing sensor assembly, facing down and moving upward and downward

962—user's right foot

962a—role on missionary body position

962b—role on back body position

962c—role female on male and on lower riding body position

963—user body indication

964—user display device

971a—second sensor assembly

972a—position mark

973a—penis holding by hand and moving upward and downward

974a—user's foot and body

975a—AR augmented reality sensor

971b—AR display

972b—AR object-AR female role

981a—third sensor assembly

981b—electromyography sensor

981c—VR display

982a—VR arm

982b—VR female role

What is claimed is:

1. A sexual need interactive platform system, which is characterized by comprising:
a communication device having a central processor and an internal memory for managing and controlling the sexual need interactive platform system;
a human-machine interaction software for providing human and machine interface interaction, when being loaded in the communication device, the central processor is used for executing the human-machine interaction software to allow a user to interact with the human-machine interaction software; and
a sensor assembly comprising a sensor having 3 or more axes and at least one feedback device, which is electrically connected to the communication device, and the sensor having 3 or more axes is worn on an active part of the user for the user to generate a sensor signal, transmits the sensor signal to the communication device, a user's activity is identified and a classification signal is generated through executing the human-machine interaction software, and then the classification signal is converted into a control signal for controlling a content of the human-machine interaction software, and a response generated by the content based on the control signal is transmitted to the feedback device for feed-backing a result of the user's activity;
wherein the communication device and the sensor assembly are connected in a wired or wireless manner.

2. A sexual need interactive platform system, which is characterized by comprising:
a content server having a content central processor and a content internal memory for providing a human-machine interaction software, which is selectable random contents;
a communication device having a central processor and an internal memory, wherein the central processor is used for executing the human-machine interaction software to allow a user to perform sexual need exercises with executed selectable contents on demand so as to control the sexual need interactive platform system; and
a sensor assembly comprising a sensor having 3 or more axes and at least one feedback device, which is electrically connected to the communication device, wherein the sensor having 3 or more axes is worn on an active part of the user for the user to generate a sensor signal, transmits the sensor signal to the communication device, a user's activity is identified and a classification signal is generated through executing the human-machine interaction software, and then the classification signal is converted into a control signal for controlling a content of the human-machine interaction software, a response generated by the content based on the control signal is transmitted to the feedback device for feed-backing a result of the user's activity;
wherein, the content server and the communication device are interconnected through an internet network.

3. The sexual need interactive platform system of claim 1 or 2, which is characterized by further comprising another software program for connecting the sensor assembly and the human-machine interaction software, wherein the classification signal is converted into a control signal for transmitting the control signal corresponding to a sensor classification result to the human-machine interaction software and for transmitting one feedback signal of the content of the human-machine interaction software to the sensor assembly, so as to be bidirectionally transmitted between the sensor assembly and the human-machine interaction software, thereby assisting the user in achieving a bilateral control effect.

4. The sexual need interactive platform system of claim 1 or 2, which is characterized in that, wherein the human-machine interaction software comes from a network, a compact disc or a flash drive or other storage devices.

5. The sexual need interactive platform system of any one of claims 1 to 2, which is characterized in that, wherein the human-machine interaction software is a software that performs exercises simulating real sex.

6. The sexual need interactive platform system of claim 2, which is characterized by further comprising an artificial intelligence server electrically connected to the internet network, and having an artificial intelligence central processor and an artificial intelligence internal memory, wherein an artificial intelligence training software is executed on the artificial intelligence central processor, and the artificial intelligence training software has a machine learning, a deep learning, a natural language and an audio conversation training functions, and trained parameters are used for various content demands of the platform;
a role generative adversarial network module, wherein the artificial intelligence training software adopts a generative adversarial network technology for being used in a platform content generation module to generate virtual roles and contents through mass audio-visual data trainings;
an artificial intelligence classification technology, which comprises a sensor signal classification module, wherein after the sensor signal is collected in a training stage, an artificial intelligence deep learning result is used for classifying the sensor signal, generating a classification parameter for being used for receiving the sensor signal in an inference stage so as to obtain a behavior classification of the user, and then the behavior classification and the content of the human-machine interaction software are combined to achieve an interactive and intuitive control manner; and
a video activity analysis module which analyzes sexual activities of the roles in a video, can convert a sexual activity process into control signals of the vibration motor and the activity structure, so that the sensor assembly generates sexual activities to allow the user to experience a sexual feeling like a main role in the screen.

7. The sexual need interactive platform system of claim 6, which is characterized in that the role generative adversarial network module further comprises an infinite audio generation sub-module which adopts Cycle GAN audio technology to simulate the voice of a first timbre by using a second timbre; and an infinite video role generation sub-module for generating various roles required by the system.

8. The sexual need interactive platform system of claim 1, which is characterized by further comprising a video activity analysis software, wherein after any sexual video such as a film, live streaming is analyzed by the video activity analysis software, all sexual behaviors within the analysis range can be recovered in real time to sexual activities through controlling the appropriate sensor assembly by the video activity analysis software, allowing sexual organs of the user to experience real feeling like a main role in the film.

9. The sexual need interactive platform system of claim 6, which is characterized by further comprising an intelligent digital lens electrically connected to the artificial intelligence server for face identification, emotion identification, behavior identification and age identification to generate a multi-modal output value, and a past video data in the artificial intelligence server is used for performing an artificial intelligence calculation to generate an optimal physiological activity condition signal.

10. The sexual need interactive platform system of claim 6, which is characterized by further comprising an intelligent speaker device electrically connected to the artificial intelligence server, wherein the intelligent speaker device converses with the user after receiving an audio conversation signal of the user from the artificial intelligence server, and then performs an artificial intelligence calculation to generate a first multimodal output value and a first identification voice-frequency conversation signal.

11. The sexual need interactive platform system of claim 6, which is characterized by further comprising a user preference and habit identification server electrically connected to the artificial intelligence server and having a user preference and habit identification central processor and a user preference and habit identification internal memory, wherein a user preference and habit identification software is executed on the user preference and habit identification central processor, for recording the user's behaviors, performing an artificial intelligence calculation, and then generating a user sexual preference and habit identification signal, so that the user preference and habit identification server generates a recommendation system based on the user's preferences and habits.

12. The sexual need interactive platform system of claim 11, which is characterized by further comprising a physician robot computer device electrically connected to the user preference and habit identification server and having a physician robot central processor and a physician robot internal memory, which is used for managing and monitoring the sexual need system while monitoring the user's physiological and medical data, so as to generate a physician robot diagnostic signal after performing an artificial intelligence calculation, and the user is informed with a sexual health diagnostic result through the intelligent speaker device.

13. The sexual need interactive platform system of claim 1 or 2, which is characterized by further comprising an audio-visual playback software for storing audio-visual data and classification parameters on the communication device, wherein when wearing the sensor assembly, hand gesture/control identification is performed by transmitting the sensor signal to the audio-visual playback software to achieve the effect of enhancing interaction with the video.

14. The sexual need interactive platform system of claim 1 or 2, which is characterized in that, wherein the sensor assembly is a first sensor assembly installed with a nine-axis sensor for detecting accelerations, movement trajectories and positions.

15. The sexual need interactive platform system of claim 1 or 2, which is characterized in that, wherein the sensor assembly is a second sensor assembly serving as a sensor for AR augmented reality, installed with a positioning mark so as to bring the video to a three-dimensional space through the communication device.

16. The sexual need interactive platform system of claim 1 or 2, which is characterized in that, wherein the sensor assembly is a third sensor assembly, which further comprises an electromyography (EMG) sensor, and an artificial intelligence algorithm is used for analyzing electromyographic signals of the electromyography sensor, so as to realize various hand gestures as fine as finger movements.

17. The sexual need interactive platform system of claim 9 or 10, which is characterized in that, wherein these multi-modal output values are output values of face identification status, emotion identification status, behavior identification status or age identification status.

18. The sexual need interactive platform system of claim 1 or 2, which is characterized in that, when the user wears the sensor assembly, it is electrically connected to a user personal health data server for transmitting the user's physiological data sensed by the sensor assembly to the user personal health data server.

19. A sexual need interactive platform system, which is characterized by comprising:
a content server equipped with a content central processor and a content internal memory for providing a human-machine interaction software, which is selectable random contents;
a first communication device having a first communication device central processor and a first communication device internal memory, wherein the first communication device central processor is used for executing the human-machine interaction software so as to manage and control the sexual need interactive platform system;
a first sensor assembly which comprises a first sensor having 3 or more axes, a first vibration motor and a first activity structure, electrically connected to the first communication device, wherein the sensor having 3 or more axes is worn on an active part of a first user for the first user to generate a first user activity signal, the first user activity signal is converted into a first activity classification signal on the central processor of the first communication device, and then converted into a first activity control signal so as to be transmitted to an internet network by the first communication device;
a second communication device, electrically connected to the first communication device, having a second communication device central processor and a second communication device internal memory, wherein the second communication device central processor is used for executing the human-machine interaction software so as to manage and control the sexual need interactive platform system; and
a second sensor assembly, which comprises a second sensor having 3 or more axes, a second vibration motor and a second activity structure, electrically connected to the second communication device, wherein the sensor having 3 or more axes is worn on an active part of a second user for the second user to generate a second user activity signal, the second user activity signal is converted into a second activity classification signal on the central processor of the second communication device, and then converted into a second activity control signal so as to be transmitted to the internet network by using the second communication device;
wherein the first communication device receives the second user activity control signal from the internet network, and then the original activity of the second user is recovered through the first activity structure; or the second communication device receives the first activity control signal from the Internet network, and then the original activity of the first user is recovered through the second activity structure;
wherein the content server, the first communication device and the second communication device are connected through the internet network.
